(19) United States
(12) Patent Application Publication
(10) Pub. No.: US 2018/0369876 A1
(43) Pub. Date: Dec. 27, 2018

(54) METHOD AND APARATUS FOR MONITORING FLUID PROPERTIES RELATING TO MEDICAL DEVICE'S CLEANLINESS

(71) Applicant: Medivators Inc., Plymouth, MN (US)

(72) Inventors: Jonathan K. Olson, Delano, MN (US); John Matta, Shoreview, MN (US); Michael Petersen, Eden Prairie, MN (US); Nick G. Bromiley, Annandale, MN (US)

(21) Appl. No.: 15/777,766

(22) PCT Filed: Nov. 15, 2016
(86) PCT No.: PCT/US2016/062099

§ 371 Date: May 21, 2018

Related U.S. Application Data
(60) Provisional application No. 62/259,468, filed on Nov. 24, 2015.

(51) Int. Cl. B08B 3/14; A61B 1/12; B08B 3/08; B08B 3/10; B01D 61/02; C02F 1/44

(52) U.S. Cl. CPC B08B 3/14; A61B 1/123; B08B 3/08; C02F 2103/003; B01D 61/025; C02F 1/441; B08B 3/102

(57) ABSTRACT

Embodiments of the present disclosure include methods and apparatuses for monitoring fluid properties relating to a medical device's cleanliness. In embodiments, an apparatus for monitoring the fluid properties relating to a medical device's cleanliness comprises at least one sensor that measures a pre-cleaning parameters of a cleaning fluid and a post-cleaning parameters of the cleaning fluid, a cleaning chamber coupled to the at least one sensor, the cleaning chamber configured to receive the medical device and clean the medical device using the cleaning fluid, and a processing device coupled to the at least one sensor. The processing device is configured to: determine a cleanliness of the medical device based on a comparison between the post-cleaning parameters and the pre-cleaning parameters; and output the cleanliness of the medical device to a display device.

### CROSS-REFERENCE TO RELATED APPLICATIONS

This application claims priority to Provisional Application No. 62/259,468 filed Nov. 24, 2015, which is herein incorporated by reference in its entirety.

### TECHNICAL FIELD

Embodiments of the present disclosure relate to a method and apparatus for cleaning of a medical device. More specifically, embodiments of the present disclosure relate to a method and apparatus for determining the cleanliness of a medical device by monitoring properties of the fluid used to clean the medical device.

### BACKGROUND

Certain medical devices are cleaned and disinfected after usage, instead of being replaced. For medical devices that are not replaced, adequate cleaning of a contaminated medical device is essential. One type of medical device that is cleaned, instead of being replaced, is an endoscope. It is current medical practice to clean and disinfect an endoscope between each endoscope examination. If an endoscope is not cleaned effectively, the effectiveness of disinfection of the endoscope may be encumbered.

### SUMMARY

Embodiments of the present disclosure determine whether a medical device is cleaned effectively. In particular, embodiments of the subject matter disclosed herein include devices and methods for monitoring fluid properties relating to a medical device's cleanliness. In embodiments, an apparatus and method, as disclosed herein, include testing a cleaning fluid, which is used to clean a medical device, before and after the cleaning fluid is used to clean medical device. In embodiments, a comparison between the tests conducted on the cleaning fluid before and after the cleaning fluid is used to clean the medical device are correlated to a medical device's cleanliness. The medical device's cleanliness may be used to determine whether the medical device is unclean, whether the medical device is in need of repairs and/or should not be further processed by disinfection. In embodiments the tests conducted on the cleaning fluid before the cleaning fluid is used to clean the medical device may be replaced with baseline measurements, as explained below. Exemplary embodiments include the following:

In an Example 1, a system for monitoring the fluid properties relating to a medical device's cleanliness comprises: at least one sensor that measures a pre-cleaning parameters of a cleaning fluid and a post-cleaning parameters of the cleaning fluid; a cleaning chamber coupled to the at least one sensor, the cleaning chamber configured to receive the medical device and clean the medical device using the cleaning fluid; a processing device coupled to the at least one sensor, the processing device configured to: determine a cleanliness of the medical device based on a comparison between the post-cleaning parameters and the pre-cleaning parameters; and output the cleanliness of the medical device to a display device.

In an Example 2, the system of Example 1, wherein when the at least one sensor measures a pre-cleaning parameters and a post-cleaning parameters of the cleaning fluid, the at least one sensor measures at least one of the following: an amount of total dissolved solid, an amount of total dissolved oxygen, an amount of total organic carbon, an amount of fluorescent agent, an amount of activated carbon, a pH and a conductance of the cleaning fluid.

In an Example 3, the system of any of Examples 1 and 2, wherein the processing device is further configured to: determine if the cleanliness of the medical device exceeds a cleanliness threshold.

In an Example 4, the system of any of Examples 1-3, wherein the processing device is further configured to: determine if the medical device is damaged.

In an Example 5, the system of any of Examples 1-4, wherein the cleaning fluid is purified using reverse osmosis before the at least one sensor measures the pre-cleaning parameters.

In an Example 6, the system of any of Examples 1-5, wherein when the sensor measures the pre-cleaning parameters and the post-cleaning parameters, the cleaning fluid is being agitated.

In an Example 7, the system of any of Examples 1-6, wherein the cleaning fluid is treated, the treatment including adding one or more chemical compositions to the cleaning fluid that react with contaminants or soil on the medical device.

In an Example 8, the system of any of Examples 1-7, wherein the medical device is an endoscope.

In an Example 9, the system of any of Examples 1-8, wherein the at least one sensor also measures at least one of the following: a pressure of the cleaning fluid when the cleaning fluid is rinsing the medical device and a flow rate of the cleaning fluid through the medical device when the cleaning fluid is rinsing the medical device.

In an Example 10, a method for monitoring the fluid properties relating to a medical device's cleanliness comprises: measuring a pre-cleaning parameters of a cleaning fluid used to cleaning a medical device; rinsing the medical device with the cleaning fluid; measuring a post-cleaning parameters of the cleaning fluid; determining a cleanliness of the medical device based on a comparison of the measured pre-cleaning parameters and the measured post-cleaning parameters; and outputting the cleanliness parameter to a display device.

In an Example 11, the method of Example 10, wherein measuring a pre-cleaning parameters and a post-cleaning parameters of the cleaning fluid comprises measuring at least one the following: an amount of total dissolved solid, an amount of total dissolved oxygen, an amount of total organic carbon, an amount of fluorescent agent, an amount of activated carbon, a pH and a conductance of the cleaning fluid.

In an Example 12, the method of any of Examples 10 and 11, further comprising: determining if the cleanliness of the medical device exceeds a cleanliness threshold.

In an Example 13, the method of any of Examples 10-12, further comprising: determining if the medical device is damaged.

In an Example 14, the method of any of Examples 10-13, further comprising: purifying the cleaning fluid using reverse osmosis before measuring a pre-cleaning parameters of the cleaning fluid.

In an Example 15, the method of any of Examples 10-14, further comprising: agitating the cleaning fluid when measuring the pre-cleaning parameters and the post-cleaning parameters.

In an Example 16, the method of any of Examples 10-15, further comprising: treating the cleaning fluid, wherein treating the cleaning fluid includes adding one or more chemical compositions to the cleaning fluid that react with contaminants or soil on the medical device.

In an Example 17, the method of any of Examples 10-16, wherein the medical device is an endoscope.

In an Example 18, the method of any of Examples 10-17, wherein when rinsing the medical device, the method further comprises measuring at least one of the following: a pressure of the cleaning fluid and the flow rate of the cleaning fluid through the medical device.

In an Example 19, an apparatus comprises: a cleaning chamber for cleaning a medical device; at least one sensor coupled to the cleaning chamber, wherein the at least one sensor measures a post-cleaning parameters of a cleaning fluid used to cleaning the medical device; and a processing device coupled to the at least one sensor, the processing device configured to: determine a cleanliness of the medical device based on a comparison between the post-cleaning parameters of the cleaning fluid and a baseline parameters of the cleaning fluid.

In an Example 20, the apparatus of Example 19, wherein the post-cleaning parameters is based on at least one of the following: an amount of total dissolved solid in the cleaning fluid, an amount of total dissolved oxygen in the cleaning fluid, an amount of total organic carbon in the cleaning fluid, an amount of fluorescent agent, an amount of activated carbon, a pH of the cleaning fluid and a conductance of the cleaning fluid.

In an Example 21, the apparatus of any of Examples 19 and 20, wherein the medical device is an endoscope.

In an Example 22, the apparatus of any of Examples 19-21, wherein the processing device is further configured to: determine if the cleanliness of the medical device exceeds a threshold.

In an Example 23, the apparatus of any of Examples 19-22, wherein the processing device is further configured to: determine if medical device is damaged.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 depicts a block diagram illustrative of a system for monitoring fluid properties relating to a medical device's cleanliness, in accordance with the embodiments of the disclosure

FIGS. 2A-2B depict an illustrative diagrammatic view an apparatus that includes a cleaning chamber for monitoring fluid properties relating to a medical device's cleanliness, in accordance with the embodiments of the disclosure.

FIG. 3 depicts an illustrative graph of cleanliness of a medical device, in accordance with embodiments of the disclosure.

FIG. 4 depicts a flow diagram depicting an illustrative method for monitoring fluid properties relating to a medical device's cleanliness, in accordance with the embodiments of the disclosure.

While the disclosed subject matter is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

As the terms are used herein with respect to ranges of measurements (such as those disclosed immediately above), “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error, differences in measurement and/or manufacturing equipment calibration, human error in reading and/or setting measurements, adjustments made to optimize performance and/or structural parameters in view of differences in measurements associated with other components, particular implementation scenarios, imprecise adjustment and/or manipulation of objects by a person or machine, and/or the like.

Although the term “block” may be used herein to connote different elements illustratively employed, the term should not be interpreted as implying any requirement of, or particular order among or between, various steps disclosed herein unless and except when explicitly referring to the order of individual steps.

### DETAILED DESCRIPTION

Embodiments of the disclosure relate to cleaning a medical device and monitoring the fluid properties used to clean the medical device. By monitoring the fluid properties used to clean the medical device, the medical device's cleanliness can be determined. The embodiments discussed below are primarily discussed in relation to an endoscope. However, the embodiments apply to other medical devices, as well.

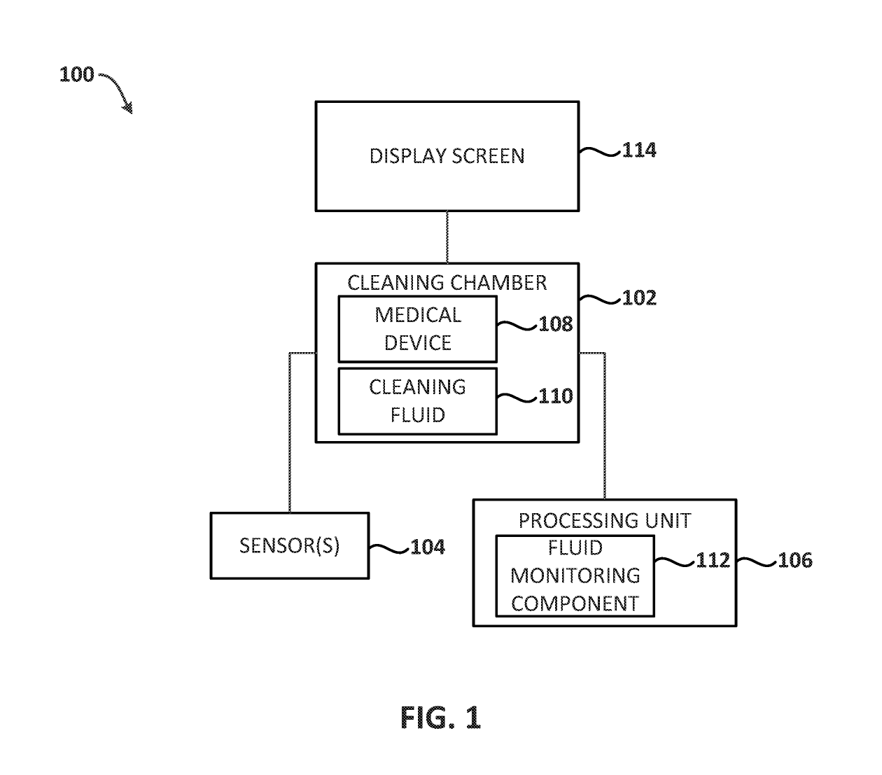

FIG. 1 depicts a block diagram illustrative of a system 100 for monitoring fluid properties relating to a medical device's cleanliness, in accordance with the embodiments of the disclosure. The illustrative system 100 includes a cleaning chamber 102, one or more sensors 104, a processing unit 106 and a display device 114. The cleaning chamber 102 is configured to receive a medical device 108 and clean the medical device 108 using a cleaning fluid 110. The cleaning chamber 102 may be designed to clean a specific type of medical device 108. For example, the cleaning chamber 102 may be specifically designed to clean an endoscope.

For endoscopes, “cleaning an endoscope” is one phase of a multi-phase process that is used to ensure an endoscope is safe for use in the next patient. In general, the multiple phases may include: a leak test phase, a pre-flushing phase, a disinfectant phase, a rinse phase and a drying phase. The embodiments discussed herein relate primarily to the pre-flushing phase (referred to herein as the “cleaning phase”). However, if one or more of the other phases are combined into a single phase, the systems and methods described herein may be performed simultaneously with another phase. Further details explaining the various phases of cleaning an endoscope are provided in U.S. Pat. No. 6,641,781 and E.P. Patent No. 0709056, which are expressly incorporated herein by reference in their entireties for all purposes.

To clean the medical device 108, the cleaning chamber 102 of the system 100 uses a cleaning fluid 110. To ensure the cleaning fluid 110 is clean, the cleaning fluid 110 may be purified using reverse osmosis, distillation, deionization and/or the like before using the cleaning fluid 110 to clean the medical device 108.

Before and after the cleaning fluid 110 is used to clean the medical device 108, one or more parameters of the cleaning fluid 110 may be measured by one or more sensors 104. Throughout the disclosure, the parameters measured before the cleaning fluid 110 is used to clean the medical device 108 may be referred to as pre-cleaning parameters and the parameters measured after the cleaning fluid 110 is used to clean the medical device 108 may be referred to as post-cleaning parameters. Additionally, the pre-cleaning and post-cleaning parameters may be referred collectively to as “cleanliness parameters.”

In embodiments, the cleanliness parameters that the sensors 104 measure may include a total amount of organic carbons included in the cleaning fluid 110, a total amount of inorganic compounds in the cleaning fluid 110 (e.g., salts), a total amount of dissolved solids in the cleaning fluid 110, an amount of total dissolved oxygen in the cleaning fluid 110, an amount of fluorescent agent in the cleaning fluid 110, an amount of activated carbon in the cleaning fluid 110, the pH of the cleaning fluid 110, and/or the conductivity of the cleaning fluid 110. Additionally, in embodiments where the medical device 108 has an interior that is flushed with the cleaning fluid 110, the sensors 104 may include measuring the flow rate of the cleaning fluid 110 through the medical device 108 and the pressure of the cleaning fluid 110 as it flushes the interior of the medical device 108. These measurements by the sensors 104 may be used to determine the medical device's 108 cleanliness. The medical device's 108 cleanliness may be used to determine whether the medical device is unclean, whether the medical device is in need of repairs and/or should not be further processed by disinfection, as described in more detail below.

In embodiments, instead of measuring one or more parameters before the cleaning fluid 110 is used to clean the medical device 108, one or more baseline parameters may be used to determine the medical device's cleanliness. In embodiments, if there is a specific valve that is providing the cleaning fluid 110 to clean the medical device 108, the cleaning fluid 110 coming from the valve may be measured at successive times and days to determine an average of the cleanliness parameters of the cleaning fluid 110 that is dispensed from the valve. These averages may be used as the baseline parameters. The cleanliness parameters measured before the cleaning fluid 110 is used to clean the medical device 108 and/or the baseline parameters may be referred to herein as “pre-cleaning parameters” and are used to determine the medical device's 108 cleanliness, as described in more detail below.

Once the pre-cleaning parameters are determined, the cleaning fluid 110 is used to clean the medical device 108. For endoscopes, the cleaning fluid 110 may be used to flush the channels of the endoscope and wash the exterior of the endoscope. Other medical devices 108 may be cleaned according to conventional methods used for the respective medical devices 108. In embodiments, the cleaning fluid 110 is water with a detergent added to the water. Reagents may also be added to the cleaning fluid 110 to help clean the medical device 108 or aid in the measurements of the pre-cleaning and post-cleaning parameters, as discussed in more detail below.

Once the medical device 108 is cleaned using the cleaning fluid 110, one or more cleanliness parameters of the cleaning fluid 110 are measured by the sensors 104. The cleanliness parameters measured after the cleaning fluid 110 is used to clean the medical device 108 may be referred to herein as “post-cleaning parameters” and are used to determine the medical device's 108 cleanliness, as described in more detail below.

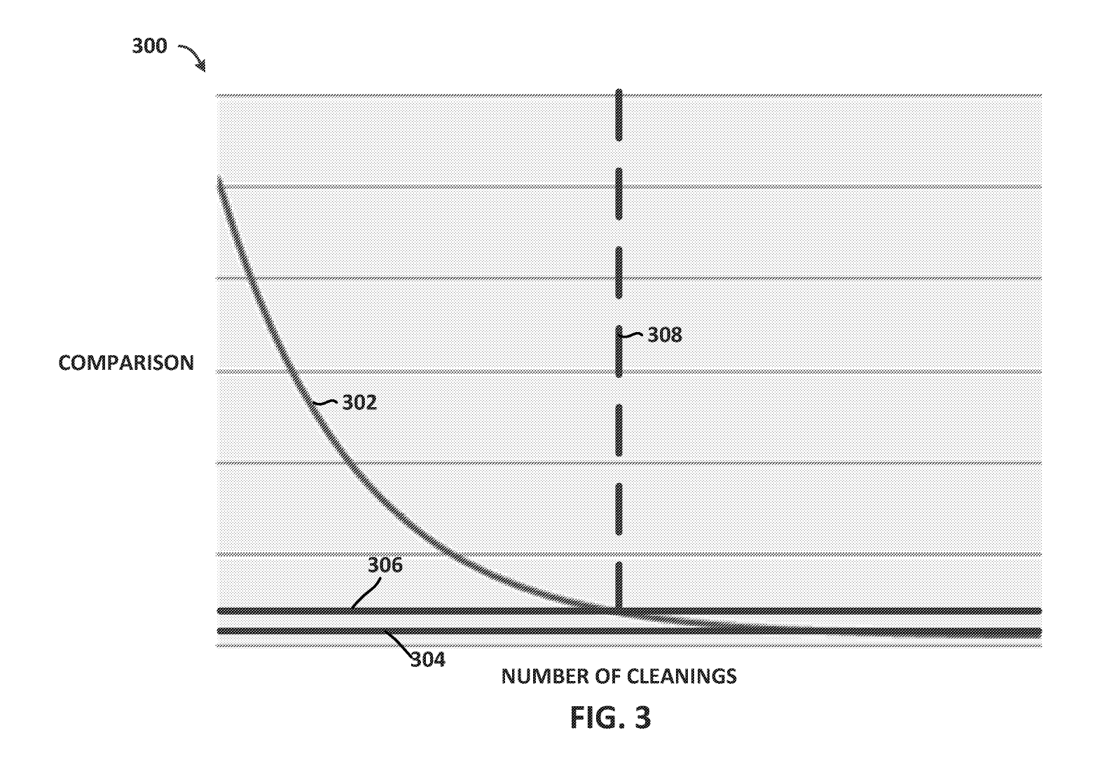

In embodiments, the processing unit 106 of the system 100 includes a fluid monitoring component 112. Based on the pre-cleaning parameters and the post-cleaning parameters, the fluid monitoring component 112 may include instructions to determine the medical device's cleanliness, as discussed below in FIG. 3. The medical device's 108 cleanliness may then be output to a display device 114 for a user of the system 100 to determine whether the medical device 108 may need to be cleaned again or if the medical device 108 may be ready to be disinfected. The display device 108 can include, but is not limited to, a CRT (cathode ray tube) display, a LCD (liquid crystal display) display, a light-emitting diode (LED) display, a plasma display and/or the like.

One of ordinary skill in the art will understand that various components such as, for example, aspects of the sensors 104, processing unit 106, the fluid monitoring component 112, and/or the display device 114, may be implemented using software, hardware, and/or firmware. Various methods of operation may be implemented as a set of instructions contained on a computer-accessible medium capable of directing a processor to perform the respective method.

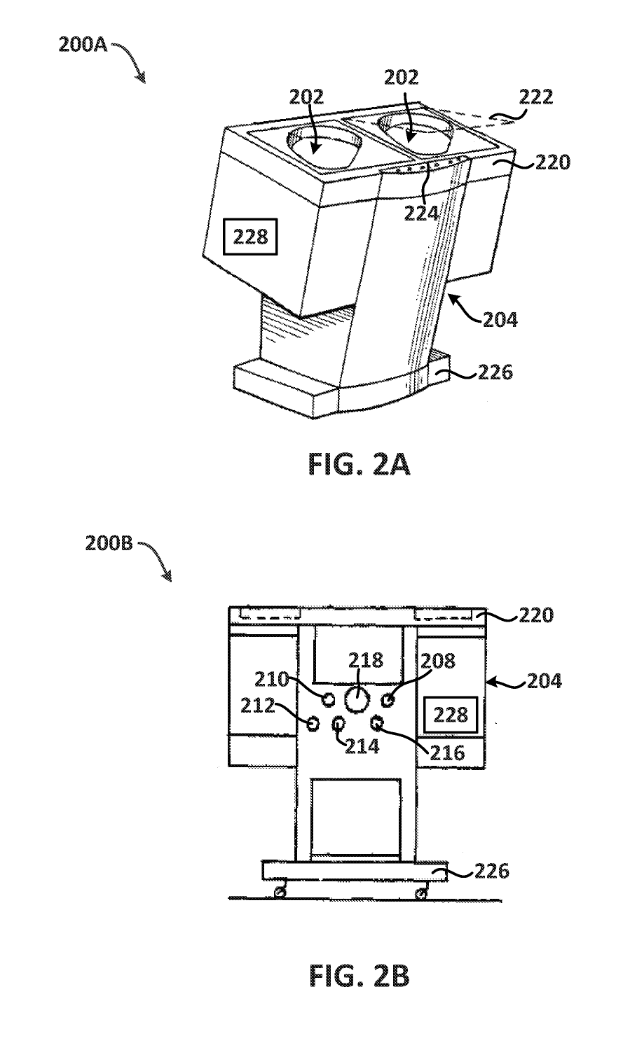

FIGS. 2A-2B depict an illustrative diagrammatic view an apparatus 200 that includes cleaning chambers 202 for monitoring fluid properties relating to a medical device's cleanliness, in accordance with the embodiments of the disclosure. This particular apparatus 200 is used to clean an endoscope. Other types of apparatuses may be used to clean other types of medical devices.

The apparatus 200 includes a housing 204, which includes cleaning chambers 202. The housing 204 is provided with connections 208-214 for cold water (e.g., water at a temperature of 20° C.), hot water (e.g., water at a temperature of 70° C.), compressed air and electricity (e.g., 220 V), an outlet 216 for spent liquids that can be connected to the sewer, and an air outlet 218 which can be connected to an extraction unit.

The housing 204 also has a top piece 220, in which two cleaning chambers 202 are accommodated, each of the two cleaning chambers 202 being able to receive an endoscope for cleaning and disinfecting. In embodiments, the capacity of each cleaning chamber 202 is approximately 5 liters. Each cleaning chamber 202 may be formed in such a way that when a cleaning chamber 202 is filled with liquid an endoscope for cleaning and disinfection is completely immersed in liquid. Each cleaning chamber 202 may be provided at the top side with a cover 222. Two endoscopes can be treated simultaneously and independently of each other. In embodiments, each cleaning chamber 202 may include a nozzle (not shown) that an endoscope can be attached to for flushing different channels of the endoscope.

The apparatus 200 may also include a control panel 224 to control the cleaning of the endoscope (e.g., the duration of the cleaning cycle, the pressure of cleaning fluid through the endoscope, the flow rate of the cleaning fluid through the endoscope and/or the like). The housing 204 may also stand on a base 226 which, as shown, may be provided with wheels.

As stated above, each cleaning chamber 202 is coupled to at least one sensor 228. The sensor 228 may be coupled to any portion of the cleaning chamber 202 where the sensor 228 is able to measure one or more pre-cleaning and post-cleaning parameters. For example, the sensor 228 may be incorporated into a recirculation loop of the cleaning chambers 202 (e.g., integrated into a nozzle or spray arm (not shown) of the cleaning chambers 202) or attached to a drain (not shown) of the cleaning chambers 202. However, these are only examples and not meant to be limiting. Depending on the location of the sensor 228, in embodiments, the cleaning chambers 202 receive the cleaning fluid after the sensor 228 measures one or more cleanliness parameters of the cleaning fluid; or, the sensor 228 measures one or more cleanliness parameters of the cleaning fluid while the cleaning fluid is in the cleaning chambers 202.

In embodiments, the sensor 228 measures one or more pre-cleaning parameters of a cleaning fluid and one or more post-cleaning parameters of the cleaning fluid. In embodiments, however, the pre-cleaning parameters may be replaced with baseline parameters, as described above. Moreover, the pre-cleaning and post-cleaning parameters that the sensor 228 is configured to measure may include, but are not limited to, the following: total organic carbon in the cleaning fluid, total inorganic compound of the cleaning fluid, total dissolved solids in the cleaning fluid, total dissolved oxygen in the cleaning fluid, an amount of fluorescent agent in the cleaning fluid, an amount of activated carbon in the cleaning fluid, pH of the cleaning fluid and conductivity of the cleaning fluid.

When measuring the pre-cleaning and post-cleaning parameters, even though 5 liters (or a different amount) of cleaning fluid may be used to clean the medical device, only a portion of the cleaning fluid may be measured by the sensor 228. In embodiments, the sensor 228 may only measure 1-2% of the total amount of cleaning fluid used to clean the medical device. For example, if 5 liters of cleaning fluid is used to clean the medical device, only 50-100 mL may be measured by the sensor 228 to determine the one or more cleanliness parameters. This is only an example, however, and not meant to be limiting. In embodiments where only a portion of the total amount of cleaning fluid, the portion being measured may be agitated to create a more homogenous mixture.

To aid in measuring one or more cleanliness parameters of the cleaning fluid or to aid in the cleaning of the endoscope, one or more reagents and/or detergents may be added to the cleaning fluid before the cleaning fluid is used to clean the medical device. Examples of reagents may include: a range of enzymatic, non-enzymatic and alkaline surfactants.

In addition, a layer of an endoscope may be impregnated with one or more agents so that if one or more of the agents are detected, it may be determined that the endoscope is damaged. For example, the biopsy/suction channel of an endoscope may have multiple layers in its construction. In embodiments, the layers of the endoscope may be made of Goa and/or Teflon. One or more of these layers (e.g., the innermost layer) may be impregnated with a fluorescent agent or an alternative agent, e.g., activated carbon. As such, if the endoscope is damaged (e.g., by an accessory) the agent used to impregnate a layer of the endoscope may be exposed to the cleaning fluid. The agent may then be detected in the cleaning parameters and it can be determined that the endoscope is damaged.

Additionally, the sensor 228 may also measure cleaning fluid flow rates going through the endoscope, pressure of the cleaning fluid as its going through the endoscope, connectivity to determine whether an endoscope is properly connected to a flushing nozzle and blockages to determine whether one or more of the channels of an endoscope is blocked. In embodiments, one or more of these measurements may affect the determination of the cleanliness of the medical device, as described below.

After one or more pre-cleaning parameters and one or more post-cleaning parameters are measured, the measurements may be transferred to a processing unit (e.g., the processing unit 106 described in FIG. 1) to determine the cleanliness of the medical device.

To determine the cleanliness of the medical device, the processing device may compare the pre-cleaning parameters to the post-cleaning parameters (referred to herein as the “measured comparison”). In embodiments, the comparison may include taking a difference between the post-cleaning and pre-cleaning parameters. The measured comparison may then be correlated to a “tested comparison.”

In embodiments, the tested comparison may be determined from one or more tests performed on a clean and/or new medical device. That is, a new and/or clean medical device may be cleaned using a cleaning fluid. The pre-cleaning and post-cleaning parameters of the cleaning fluid used to clean the new and/or clean medical device may be measured by sensors similar to the sensors 228 above. After which, a comparison between the pre-cleaning and post-cleaning parameters may be determined. This comparison, referred to as the “tested comparison,” may then be used to signify what the measured comparison should approximately be to determine that a medical device is clean and/or damaged.

FIG. 3 depicts an illustrative graph 300 of cleanliness of a medical device, in accordance with embodiments of the disclosure. In embodiments, the vertical axis represents a comparison between the pre-cleaning parameters and the post-cleaning parameters, as described above. The comparison between the pre-cleaning parameters and the post-cleaning parameters may include comparisons of total dissolved solids in a cleaning fluid, total dissolved oxygen in a cleaning fluid, total organic carbons in a cleaning fluid, total inorganic compounds in a cleaning fluid, an amount of fluorescent agent in the cleaning fluid, an amount of activated carbon in the cleaning fluid, an amount of fluorescent agent in the cleaning fluid, an amount of activated carbon in the cleaning fluid, pH of the cleaning fluid, conductivity of the cleaning fluid and/or the like that are measured pre-cleaning and post-cleaning of a medical device. As shown, the comparison is a function of the number of cleanings of the medical device.

In the illustrated embodiment, the graph 300 includes three curves: a measured comparison curve 302, a tested comparison curve 304 and a threshold comparison curve 306. In embodiments, the measured comparison curve 302 may represent a comparison of the pre-cleaning and post-cleaning parameters for a cleaning fluid that was used to clean a medical device, such that the medical device was used in a medical procedure on a patient. In embodiments, the measured comparison may decrease with subsequent cleanings. For example, the first time a medical device is cleaned after the medical device was used in a medical procedure, the cleaning fluid may remove a significant amount of solids and/or organic carbon from the medical device during the cleaning process. As such, a comparison between the pre-cleaning parameters and post-cleaning parameters may be significant. After subsequent cleanings, however, the comparison between the pre-cleaning parameters and the post-cleaning parameters may decrease, as shown in the graph 300, since there are less solids and organic carbons to be removed from the medical device. Accordingly, the measured comparison curve 302 may decrease as a function of the number of cleanings of the medical device.

The tested comparison curve 304 shown in graph 300 may represent a comparison of the pre-cleaning and post-cleaning parameters for a cleaning fluid that was used to clean a medical device that was either clean and/or new. In embodiments, the tested comparison may not decrease with subsequent cleanings. For example, with each subsequent cleaning, a comparison of the pre-cleaning and post-cleaning parameters for a cleaning fluid that is used to clean the new and/or clean medical device may not change significantly because the new and/or clean medical device may not include substances from a patient that were collected on the medical device during a medical procedure. As such, the solids and organic carbons removed from the new medical device may be zero.

While the solids and organic carbons removed from the new medical device may be zero, the comparison for the tested comparison curve 304 may be non-zero in embodiments. For example, inorganic compounds may be removed from the medical device by the cleaning fluid during the cleaning of the medical device. As such, the inorganic compounds in the cleaning fluid may be slightly greater after the cleaning of the medical device than before cleaning the medical device. In embodiments, however, the comparison may not change between each subsequent cleaning since roughly the same amount of inorganic compounds may be removed from the medical device by the cleaning fluid with each cleaning. Accordingly, the tested comparison curve 304 may be substantially flat, but non-zero.

In embodiments, the measured comparison curve 302 and the tested comparison curve 304 may be affected by the pH of the cleaning fluid and/or the conductivity of the cleaning fluid. As such, the comparison curves 302, 304 may be adjusted depending on the measured pH and/or conductivity of the cleaning fluid. As an example, assume the tested comparison curve 304 was determined using a cleaning fluid that had a first conductivity. Further assume the measured comparison curve 302 was determined using a cleaning fluid that had a second conductivity that was higher than the first conductivity. In this example, more inorganic compounds may be removed from the medical device when the conductivity of the cleaning fluid is higher. Accordingly, the measured comparison curve 302 may be adjusted down to account for the higher conductivity of the cleaning fluid, due to the higher amount of inorganic compounds that are removed from the medical device. Similar adjustments may be made depending on the pH of the cleaning fluid. In embodiments, however, the pH of the cleaning fluid may be continuously monitored and controlled, so that the pH of the cleaning fluid remains substantially constant (e.g., +/−1%, 2%, 5%, 10%).

After a measured comparison of the cleaning fluid is determined, to determine whether a medical device is clean, the measured comparison curve 302 may have to be within a certain threshold of the tested comparison curve 304. As such, a threshold curve 306, which is within a certain percentage of the tested comparison curve 304, is shown. As an example, the threshold curve 306 may be +2% of the tested comparison curve 304. In embodiments, once the measured comparison curve 302 intersects the threshold curve 306 or is between the threshold curve 306 and the tested comparison curve 304 (i.e., the portion of the graph 300 to the right of line 308), the medical device may be determined to be clean. If, however, the measured comparison curve 302 does not intersect the threshold curve 306 or is not between the threshold curve 306 and the tested comparison curve 304 (i.e., the portion of the graph 300 to the left of line 308), then the medical device may be determined not to be clean. In embodiments, if the medical device is determined not to be clean, the medical device may be cleaned again until the measured comparison curve 302 either intersects the threshold curve 306 or is between the threshold curve 306 and the tested comparison curve 304.

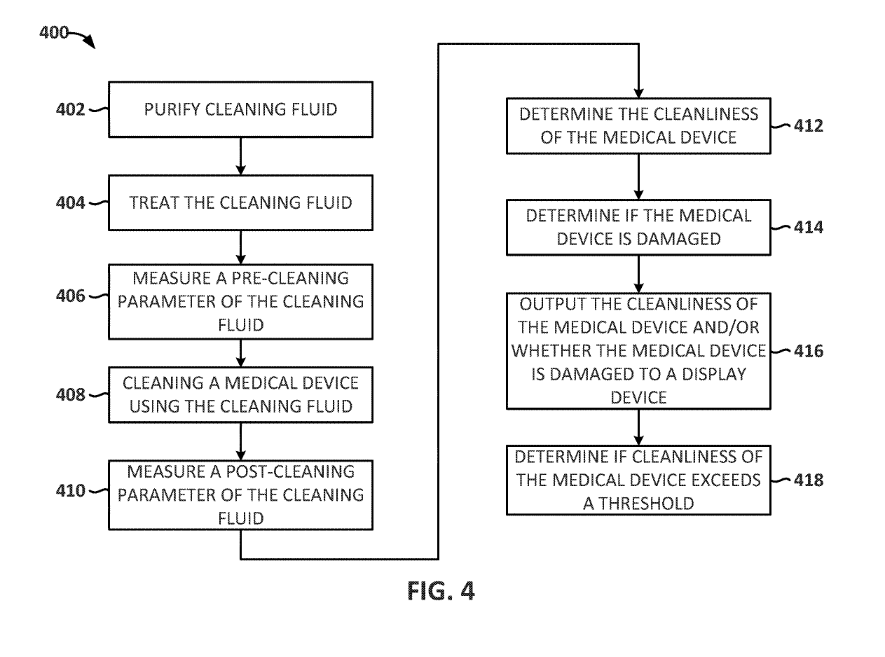

FIG. 4 depicts a flow diagram depicting an illustrative method 400 for monitoring fluid properties relating to a medical device's cleanliness, in accordance with the embodiments of the disclosure. In embodiments, method 400 includes purifying a cleaning fluid (block 402). In embodiments, the cleaning fluid may be purified to prevent contamination of the medical device and/or to aid in the measurement of the pre-cleaning and post-cleaning parameters. The cleaning fluid may be purified, for example, by reverse osmosis.

Method 400 may also include treating the cleaning fluid (block 404). As described above, the cleaning fluid may be treated to aid in the measurement of the pre-cleaning and post-cleaning parameters and/or to aid in the cleaning of the medical device. To treat the cleaning fluid, reagents and/or detergents may be added to the cleaning fluid.

Method 400 may also include measuring a pre-cleaning parameter of the cleaning fluid (block 406). As described above, measuring a pre-cleaning parameter may include measuring one or more of the following parameters of the cleaning fluid: total organic carbon in the cleaning fluid, total inorganic compound of the cleaning fluid, total dissolved solids in the cleaning fluid, total dissolved oxygen in the cleaning fluid, an amount of fluorescent agent in the cleaning fluid, an amount of activated carbon in the cleaning fluid, pH of the cleaning fluid and conductivity of the cleaning fluid. Additionally, in embodiments where the cleaning fluid is sent through a medical device, measuring pre-cleaning parameters may include measuring: fluid flow rates going through the medical device, pressure of the cleaning fluid as its going through the medical device, connectivity to determine whether the medical device is properly connected to a flushing nozzle and blockages to determine whether one or more of the channels of the medical device is blocked.

In embodiments, instead of measuring pre-cleaning parameters, baseline parameters may be used in method 400.

Method 400 also includes cleaning a medical device using the cleaning fluid (block 408). As described above, cleaning the medical device may depend on the type of medical device that is being cleaned. For example, if the medical device is an endoscope, cleaning the medical device using the cleaning fluid may include flushing the channels of the endoscope and cleaning the exterior of the endoscope. An apparatus such as the one described above in FIG. 2 may be used to clean the endoscope with the cleaning fluid.

Method 400 may also include measuring a post-cleaning parameter of the cleaning fluid (block 410). The post-cleaning parameter may be the same or similar to the pre-cleaning parameter.

After measuring a post-cleaning parameter, method 400 may include determining a cleanliness of the medical device (block 412). In embodiments, the cleanliness of the medical device may be based on a comparison between the pre-cleaning parameters and the post-cleaning parameters. The comparison may include determining a difference between the post-cleaning parameters and the pre-cleaning parameters. Based on the comparison between the pre-cleaning and post-cleaning parameters, the cleanliness of the medical device may be determined, e.g., as described above in FIG. 3.

Method 400 may also include determining whether the medical device is damaged (block 414). In embodiments, the determination of whether a medical device is damaged may be based on detecting one or more agents that were used to impregnate a layer of the medical device, as described above. For example, a layer of an endoscope may be impregnated with one or more agents so that if one or more of the agents are detected, it may be determined that the endoscope is damaged. In embodiments, one or more of these layers of the endoscope (or other medical device) may be impregnated with a fluorescent agent or an alternative agent like activated carbon. As such, if the endoscope is damaged (e.g., by an accessory) the agent used to impregnate a layer of the endoscope may be exposed to the cleaning fluid. The agent may then be detected in the cleaning parameters and it can be determined that the endoscope is damaged.

Method 400 may also include outputting the cleanliness of the medical device to a display device and outputting if the medical device is damaged to a display device (block 416). In embodiments, this may help a user determine whether the medical device can be used in another patient, whether the medical device needs to be cleaned again and/or whether the medical device needs to be repaired or replaced.

In embodiments, method 400 also includes determining if the cleanliness of the medical device exceeds a threshold (block 418). In embodiments, and similar to above, this may help a user determine whether the medical device needs to be cleaned again and/or is safe to use in another patient.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

1. A system for monitoring the fluid properties relating to a medical device's cleanliness, the apparatus comprising:
at least one sensor that measures a pre-cleaning parameters of a cleaning fluid and a post-cleaning parameters of the cleaning fluid;
a cleaning chamber coupled to the at least one sensor, the cleaning chamber configured to receive the medical device and clean the medical device using the cleaning fluid;
a processing device coupled to the at least one sensor, the processing device configured to:
determine a cleanliness of the medical device based on a comparison between the post-cleaning parameters and the pre-cleaning parameters; and
output the cleanliness of the medical device to a display device.

2. The system of claim 1, wherein when the at least one sensor measures a pre-cleaning parameters and a post-cleaning parameters of the cleaning fluid, the at least one sensor measures at least one of the following: an amount of total dissolved solid, an amount of total dissolved oxygen, an amount of total organic carbon, an amount of fluorescent agent, an amount of activated carbon, a pH and a conductance of the cleaning fluid.

3. The system of claim 1, wherein the processing device is further configured to: determine if the cleanliness of the medical device exceeds a cleanliness threshold.

4. The system of claim 1, wherein the processing device is further configured to: determine if the medical device is damaged.

5. The system of claim 1, wherein the cleaning fluid is purified using reverse osmosis before the at least one sensor measures the pre-cleaning parameters.

6. The system of claim 1, wherein when the sensor measures the pre-cleaning parameters and the post-cleaning parameters, the cleaning fluid is being agitated.

7. The system of claim 1, wherein the cleaning fluid is treated, the treatment including adding one or more chemical compositions to the cleaning fluid that react with contaminants or soil on the medical device.

8. The system of claim 1, wherein the medical device is an endoscope.

9. The system of claim 1, wherein the at least one sensor also measures at least one of the following: a pressure of the cleaning fluid when the cleaning fluid is rinsing the medical device and a flow rate of the cleaning fluid through the medical device when the cleaning fluid is rinsing the medical device.

10. A method for monitoring the fluid properties relating to a medical device's cleanliness, the method comprising:
measuring a pre-cleaning parameters of a cleaning fluid used to cleaning a medical device;
rinsing the medical device with the cleaning fluid;
measuring a post-cleaning parameters of the cleaning fluid;
determining a cleanliness of the medical device based on a comparison of the measured pre-cleaning parameters and the measured post-cleaning parameters; and
outputting the cleanliness parameter to a display device.

11. The method of claim 10, wherein measuring a pre-cleaning parameters and a post-cleaning parameters of the cleaning fluid comprises measuring at least one the following:
an amount of total dissolved solid, an amount of total dissolved oxygen, an amount of total organic carbon, an amount of fluorescent agent, an amount of activated carbon, a pH and a conductance of the cleaning fluid.

12. The method of claim 10, further comprising: determining if the cleanliness of the medical device exceeds a cleanliness threshold.

13. The method of claim 10, further comprising: determining if the medical device is damaged.

14. The method of claim 10, further comprising: purifying the cleaning fluid using reverse osmosis before measuring a pre-cleaning parameters of the cleaning fluid.

15. The method of claim 10, further comprising: agitating the cleaning fluid when measuring the pre-cleaning parameters and the post-cleaning parameters.

16. The method of claim 10, further comprising: treating the cleaning fluid, wherein treating the cleaning fluid includes adding one or more chemical compositions to the cleaning fluid that react with contaminants or soil on the medical device.

17. The method of claim 10, wherein the medical device is an endoscope.

18. The method of claim 10, wherein when rinsing the medical device, the method further comprises measuring at least one of the following: a pressure of the cleaning fluid and the flow rate of the cleaning fluid through the medical device.

19. An apparatus comprising:
a cleaning chamber for cleaning a medical device;
at least one sensor coupled to the cleaning chamber, wherein the at least one sensor measures a post-cleaning parameters of a cleaning fluid used to cleaning the medical device; and
a processing device coupled to the at least one sensor, the processing device configured to: determine a cleanliness of the medical device based on a comparison between the post-cleaning parameters of the cleaning fluid and a baseline parameters of the cleaning fluid.

20. The apparatus of claim 19, wherein the post-cleaning parameters is based on at least one of the following: an amount of total dissolved solid in the cleaning fluid, an amount of total dissolved oxygen in the cleaning fluid, an amount of total organic carbon in the cleaning fluid, an amount of fluorescent agent, an amount of activated carbon, a pH of the cleaning fluid and a conductance of the cleaning fluid.

21. The apparatus of claim 19, wherein the medical device is an endoscope.

22. The apparatus of claim 19, wherein the processing device is further configured to: determine if the cleanliness of the medical device exceeds a threshold.

23. The apparatus of claim 19, wherein the processing device is further configured to: determine if medical device is damaged.
